(19) United States
(12) Patent Application Publication
(10) Pub. No.: US 2010/0010595 A1
(43) Pub. Date: Jan. 14, 2010

(54) APPARATUS FOR THERMAL TREATMENT OF TISSUE

(75) Inventor: Jan Willem Bruggers, Eindhoven (NL)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 12/439,256

(22) PCT Filed: Aug. 23, 2007
(86) PCT No.: PCT/IB2007/053370

§ 371 Date: Feb. 27, 2009

(30) Foreign Application Priority Data
Aug. 30, 2006 (EP) 06119781.0

(51) Int. Cl. A61B 5/055; A61N 7/02

(52) U.S. Cl. 607/96; 601/3

(57) ABSTRACT

An apparatus for thermal treatment of tissue comprises a compact manipulator (37) for positioning and orienting an energy radiator (50) for heat treatment of biological tissues. The energy radiator (50) is adapted to emit energy in the direction of a focal axis into a focal volume. The manipulator (37) can adjust the position and orientation of a suspension body (20) in a plane. The plane wherein the suspension body can be maneuvered is parallel to a support face of the apparatus. The energy radiator (50) is suspended from the suspension body (20). The energy radiator (50) can be manipulated with five independent degrees of freedom relative to said support face.

### FIELD OF THE INVENTION

The invention relates to an apparatus for thermal treatment of tissue, provided with a support face for supporting at least a portion of a patient's body and comprising an energy radiator mounted to a holder, for directing energy along a focusing axis into a focal volume, and a manipulator comprising a manipulator transmission unit including a suspension body, a transmission driver unit mounted to the manipulator transmission unit, the transmission driver unit comprising at least one transmission driver and the holder suspended from the suspension body, wherein the manipulator transmission unit is driveable by the transmission driver unit and the holder is driveable by the manipulator transmission unit.

### BACKGROUND OF THE INVENTION

An apparatus for thermal treatment of tissue is known from the international application WO 2005/107870. The apparatus described in application WO 2005/107870 is claimed to be suitable for treating tumors in breast tissue by means of High Intensity Focused Ultrasound (HIFU). In HIFU systems the ultrasound energy generated by an energy radiator is focused into a small focal volume at the specific target locations of for example cancerous tissue. During treatment, the beam of focused energy penetrates through tissue and causes localized temperature rises in a well-defined region being the focal volume. Thus, ultrasound beams are focused on a tissue, and due to the energy deposition at the focus, temperature within the tissue rises to a level, completely destroying it. The temperature rise produces preferably well-defined regions of protein denaturisation, irreversible cell damage and necrosis. A single exposure of focused ultrasound energy is called a sonication. Sonication is a process of dispersing, disrupting or deactivating biological materials by the use of sound waves. Multiple sonications are necessary to ablate the targeted tissue. Tight focusing is needed to limit the ablation only to the targeted location of a patient's lesion, myoma, uterine fibroid or the like. This technology can achieve precise ablation of diseased tissue, if the procedure is guided and controlled using Magnetic Resonance Imaging (MRI). Applying power to a patient needs planning, targeting of the energy and monitoring of the energy delivery. No energy must be scattered or dissipated unnecessarily in risky regions of e.g. nerves or vital organs. In general, the energy is emitted in an aiming direction along a focusing axis into a focal volume located at a focal distance from the energy radiator. The apparatus described in WO 2005/107870 relates to a device for positioning an energy-generating means of an assembly for heat treatment of biological tissues. Positioning of the energy-generating means of WO 2005/107870 is enabled in a plane between the bottom section of an MRI scanning apparatus and a support holding the patient. The energy-generating means of WO 2005/107870 are adapted to emit energy along a focusing axis, wherein the focusing axis is oriented substantially parallel to the coronal plane and the focal volume is positioned outside the torso while avoiding the regions inside the torso underneath the breast tissue. The coronal plane separates the human body into a ventral portion and a dorsal portion. The coronal plane is perpendicular to the median plane that separates the body into a left and right side. The device of WO 2005/107870 avoids emitting energy parallel to the median plane into risky regions accommodating vital organs. For that purpose the positioning device of WO 2005/107870 comprises an energy-generating means suspended from an annular frame. The device of WO 2005/107870 enables two perpendicular translations of the energy-generating means parallel to the coronal plane of the patient, in WO 2005/107870 indicated as T1 and T2. Perpendicular rails are provided to enable the perpendicular translations T1 and T2 of a suspension frame. The positioning device of WO 2005/107870 enables two rotations of the energy-generating means, in WO 2005/107870 indicated as R1 and R2. Thus, the maneuverability of the energy-generating means of WO 2005/107870 comprises two translations T1 and T2 and two rotations R1 and R2 to position and orient the energy-generating means relative to the support holding the patient.

However, if treatment is needed of tissue, located inside the torso underneath the breast cover or if treatment aims at prostate ablation or in case of treatment of the uterus, the component of the aiming direction of the energy transversal to the coronal plane is relatively large compared to the component of the aiming direction comprised by the coronal plane. To minimize and preferably eliminate the risks of emitting energy into unintended regions of e.g. the inner torso, accommodating vital organs or nerves or the like, the energy radiator should be positioned and oriented with high precision and high reproducibility relative to the tissue to be treated. Accurate positioning of the energy radiator or transducer is important to localize the focal volume only at the targeted tissue. Accurate orientation of the energy radiator is essential to align the focusing axis of the energy emission with a permissible path to the targeted tissue to get round possibly risky regions, vital organs, nerves and the like. Use can be made of MRI equipment to give feedback to the operator on the location of the temperature rise relative to the region of the disorder to be treated. The positioning of the focal volume and the orientation of the focusing axis should be realized by a compact device to enable treatment of tissue in combination with diagnostic equipment as MRI. This kind of equipment provides only limited space to mount and operate the positioning device. As is commonly known, the space that is available to a patient inside the bore or scanning area of MRI equipment is very limited and this may cause distress especially to patients suffering from claustrophobia. The positioning device of application WO 2005/107870 is not apt for precise positioning and alignment of the energy penetrating into regions of the body inside the torso, because the positioning device of application WO 2005/107870 is arranged to focus the energy in a plane mainly parallel to the coronal plane outside the torso and through the beast tissue covering the torso and not for positioning the focal volume and the energy generating means along a direction substantially perpendicular or transverse to the coronal plane into the torso. A further problem of the device of application WO 2005/107870 is that the device is inherently space consuming because of stacking of components as will be explained hereafter. An annular frame is positioned between a set of rails to enable translation T2. The device of application WO 2005/107870 comprises two elongated bodies to enable translation T1. The bodies need to be elongated because cranks should remain outside the bore of the MRI to be accessible for manual operation. Also if electric equipment is used to drive said cranks the electric equipment must remain outside the bore of the MRI to prevent interference and disturbance of the magnetic field of the equipment with the magnetic field generated and interpreted by the MRI equipment and used to give feedback for guidance and control of the position and orientation of the energy-generating means. Thus, in the device of application WO 2005/107870 four components are needed to enable two translations T1 and T2. If the positioning range of the annular frame is maximized, the range of possible translations T1 and T2 should be maximized. This is commonly realized by stacked straight-guides. In case of maximization of T1 and T2 in the device of application WO 2005/107870, it is not possible to position the length axes of said rails in the same plane as the plane defined by the length axes of said elongated bodies, because the rails cannot intersect with the elongated bodies. For this reason, the plane containing the length axes of the rails should have a distance to the plane containing the length axes of the elongated bodies. The distance needed to stack the rails and bodies in different planes is conflicting with the requirement of a flat and compact device, to fit into the narrow bore of an MRI apparatus without unacceptable limitation of space that remains available for patients.

### SUMMARY OF THE INVENTION

It is an object of the present invention to provide an apparatus for thermal treatment of tissue of the kind set forth in the opening paragraph, which can accurately position and orient the energy radiator in a space saving way with five independent degrees of freedom for substantially positioning the focal volume of the energy along a focusing axis.

With the apparatus for thermal treatment of tissue of the invention this object is realized in that the manipulator transmission unit has a first transmission subunit for translating and rotating the suspension body in a plane substantially parallel to the support face and has a second transmission subunit for moving the energy radiator along the focusing axis and for rotating the energy radiator around two distinct axes perpendicular to the focusing axis.

To analyze and characterize the performance of a positioning device use will be made of the degrees of freedom of a rigid body. The degrees of freedom of a rigid body are the set of independent translations and rotations that specify completely the position and orientation of the rigid body relative to a coordinate system. The translations of the rigid body represent the ability of the rigid body to move in each of the three dimensions. The rotations of the rigid body represent the ability of the rigid body to change angle around the three perpendicular axes characterizing the three dimensions. Thus, a rigid body can have a maximum of six independent degrees of freedom. The energy generating means of the device of application WO 2005/107870 has two degrees of freedom R1 and R2 relative to the annular frame from which said means is suspended and the annular suspension frame has two degrees of freedom T1 and T2 relative to its support. The manipulator transmission of the invention has a first subunit for translating and rotating the suspension body in a plane substantially parallel to the support face instead of the rails and elongated bodies of the device of application WO 2005/107870. The device of application WO 2005/107870 uses four components, being two rails and two elongated bodies. The components, needed in the first subunit to realize the translation and the rotation of the suspension body, may be arranged, such that inherently little mounting space is needed to realize a compact apparatus according to the invention. As a further advantage, it can be mentioned that with the apparatus according to the invention no stacking of components is needed to position the suspension body. The energy radiator is suspended from the suspension body. The second transmission subunit can move the energy radiator along the focusing axis and can rotate the energy radiator around two distinct axes perpendicular tot the focusing axis. Thus, the energy radiator has three independent degrees of freedom relative to the suspension body. The suspension body has two independent degrees of freedom relative to the support face. The energy radiator of the invention can thus be positioned and oriented relative to the support face with five independent degrees of freedom. Three translations are possible to position the focal volume and two rotations are possible to orient the focal axis relative to the support face. However, to provide maximum maneuverability to the radiator a maximum of six degrees of freedom could be realized. Realization of six independent degrees of freedom goes at the expense of a considerable amount of hardware. The energy is usually emitted in a volume enclosed by a surface of substantially conical shape. The focusing axis is an axis of rotation symmetry with respect to said conical surface. Because of said rotation symmetry around the focusing axis the rotation of the energy distribution around the focusing axis is considered less important than the rotation around two distinct axes perpendicular to the focusing axis. For this reason the hardware needed to realize a rotation of the radiator around the focal axis is omitted and the radiator can be maneuvered with five independent degrees of freedom comprising the ability to move in a direction transversal to the support face. The possibility of translation perpendicular to the support face enables treatment of tissue inside the human torso underneath the beast tissue.

Because the invention inherently leads to compact embodiments, more design freedom is created to optimize the geometry of the construction with respect to stiffness of the construction. Stiffness of the device enables accurate and reproducible maneuvering of the radiator. Deformation in general leads to a compliant construction that is difficult to control via feedback from the MRI. The radiator will be in the proximity of the patient's disorder, i.e. inside the bore of the MRI, while the motor will be positioned outside the detecting volume of the scanning equipment to prevent interference of the magnetic field produced by e.g. a driving motor with magnetic resonance signals to be detected by the MRI equipment. Several transmission parts are needed to bridge the distance between the motor and the radiator. A geometrically stiff construction enables the use of materials that do not necessarily have a high Young's modulus or modulus of elasticity. Furthermore, these parts should be made of materials having magnetic properties that are suitable for use in equipment for magnetic resonance imaging. A wide class of synthetic resins have magnetic properties suitable for use in magnetic resonance imaging. These synthetic resins can be reinforced with fibres to improve their mechanical strength. However, said resins do not necessarily have a high Young's modulus. For this reason it is very advantageous, that the geometrical stiffness of the construction enables the use of non-magnetic materials as synthetic resins possibly reinforced with fibres. The geometrical stiffness enables the use of these relatively compliant materials in the apparatus of the invention. Very suitable for application is a material called Werkstoff “S”®. Besides having suitable magnetic properties it has good resistance against wear and against chemicals and does retain its shape in the presence of water because it does not combine with water. If Werkstoff “S”® is used, the mechanism to position the radiator is made water-resistant and the energy radiator can be submerged into water to transmit its ultrasound waves. The sliding properties are favorable, which is desirable for good mechanical hysteresis. Suitable magnetic properties do however not necessarily exclude a high modulus of elasticity or Young's modulus, as in the case of ceramic materials. In the components loaded with bending moments ceramic materials can be used, because they retain shape, are magnetically compatible with magnetic resonance imaging and have a high modulus of elasticity. Among the ceramic materials the material category of oxides comprises materials as Aluminium oxide (Al2O3) and zinc oxide (ZnO). Said materials are suitable for use in preferred embodiments of the apparatus. Also the material category of carbides is suitable for use. Silicon carbide (SiC) can be mentioned as example of a material that is widely commercially available. Also more design freedom is created to optimize friction of the construction. High friction in combination with low stiffness causes mechanical hysteresis. Mechanical hysteresis manifests by a lag between displacement or rotation of the holder and a change in angle of rotation of a driver. This lag between action of the driver and response of the holder hinders accurate control of the positioning system via feedback provided by the MRI system. The overall ratio of stiffness of the device to friction in the device must preferably be high to prevent suffering from hysteresis. Because the invention inherently leads to compact embodiments with a limited number of stiff components, the friction in the construction can be optimized more easily. On the one hand the number of contacts is limited because of the limited number of components and on the other hand the components can be designed stiff as explained above. Stiff components allow for less deformation of the components, leading to well-defined contact situations between cooperating components. Well-defined contact between components leads to a better control of friction between components.

An embodiment of the apparatus according to the invention is defined in that the suspension body comprises distant portions and the first transmission subunit comprises moveably guided abutments and abutment guides, each of the distant portions being rotatably connected to at least one of the moveably guided abutments and each moveably guided abutment being guidably supported by at least one of the abutment guides. If the suspension is rotatably connected to a moveably guided body, pretension and friction can be minimized in the connection between the distant parts of the suspension body and the moveably guided abutments and between the moveably guided abutments and the abutment guides. This is advantageous for positioning accuracy and reproducibility as explained before. Connecting distant portions instead of close portions of the suspension body to separate moveably guided abutments is beneficial for the accuracy attainable in the rotation of the suspension body in a plane substantially parallel to the support face.

An embodiment of the apparatus according to the invention is defined in that the first transmission subunit comprises at least one transmission body for cooperation with one of the moveably guided abutments and being coupled to a separate transmission driver of the transmission driver unit. In the case of a rotatable transmission body, the transmission body can be arranged to convert a rotation into a guided translation of the moveably guided abutment. The moveably guided abutment can be positioned in a range of positions along its abutment guide. By driving the moveably guided abutment by a rotatable transmission body the total length of the system between the separate transmission driver and the abutment guide can be confined to a fixed length. Also a slideable transmission body can be used as e.g. a hydraulic cylinder. The hydraulic cylinder can be operated with water and the water can be pressurized by a hydraulic pump as embodiment of a transmission driver. The rotatable transmission body can be replaced by a slideable component, arranged such that the distance between the motor and the moveably guided abutment can be changed, as for example by a rack from a rack and pinion combination. The transmission body can also be a gear-drive, a belt-drive or a chain-drive. A further advantage of the transmission body is that the transmission driver unit may be kept outside the scanning volume to prevent possible interference of electromagnetic fields of the driver and the MRI device, if applied in an MRI device, or to be accessible for manual operation. A further advantage of a rotatable transmission body is, that it is easy to mount a measuring device to the transmission body to obtain feedback on the number of revolutions of the driver and the position of the guided body.

An embodiment of the apparatus according to the invention is defined in that the distant portions each have a first axis of rotation with respect to the moveably guided abutment to which the respective distant portion is connected and wherein the transmission body cooperating with the respective moveably guided abutment comprises a first threaded portion, said portion having a first length axis, said first length axis intersecting with said first axis of rotation. The first threaded portion may have a ridge of for example a helical or spiral shape cooperating with the moveably guided abutment. One rotation of the transmission body is transmitted via the first threaded portion into a translation of the moveably guided abutment over a defined length referred to as the pitch. Application of a small pitch is favorable for accurate adjustment of the moveably guided abutment along its abutment guide and for accurate positioning and rotation of the suspension body. Because of the intersection of said axes no side forces or bending moments are introduced on the first threaded portion and the transmission body comprising said first threaded portion. Side forces and bending moments lead to relatively large deformations compared to the deformations associated with pure push and pull loads. As explained above, large deformations are detrimental for accuracy and reproducibility of positioning of the suspension body.

An embodiment of the apparatus according to the invention is defined in that at least part of a moveably guided abutment is elastically deformed to establish pretension between the moveably guided abutment and the first threaded portion comprised by the transmission body cooperating with the respective moveably guided abutment. By controlled pretension, the mechanism can be kept free from backlash. A possible play resulting from loose connections between gears or other mechanical elements may cause a sudden or violent backward whipping motion. Such motion is harmful for positioning the radiator with a positioning accuracy in the order of magnitude of 0.1 mm.

An embodiment of the apparatus according to the invention is defined in that the holder comprises three levers, moveably suspended from the suspension body. The energy radiator preferably should not be deformed. The energy radiator may contain a number of components that can suffer from mechanical strain. It is desired, that the holder positions the energy radiator in a statically determinate way, such that the static equilibrium equations are sufficient for determining the internal forces in the holder and the reaction forces on the energy radiator. A statically determinate structure as e.g. the radiator can be defined as a structure where, if it is possible to find internal actions in equilibrium with external loads, those internal actions are unique. In general six equations are needed to establish the static equilibrium of a structure in general and of the radiator in particular. Said six equations define the magnitude of six independent external forces exerted on the radiator. The three levers comprised by the holder exert these three external forces. Each lever may e.g. exert two perpendicular forces on the radiator. By applying three levers it is possible to hold the radiator with minimal deformation of the radiator. Each of the three levers may be moveably suspended from the suspension body such that it can pivot around a pivoting point on a pivoting axis, the pivoting point and the pivoting axis being fixed relative to the suspension body. Besides its pivoting point a lever has two more characterizing points, viz. the two lever ends, the lever ends being different from the pivoting point. In the lever ends the lever is connected to other components. One end of the lever may be rotatably connected to the radiator, while the other end can be rotatably connected to a mechanism comprised by the second transmission subunit. The end of the lever, connected to the radiator will be referred to as the radiator end. The end of the lever, connected to the mechanism comprised by the second transmission subunit will be referred to as the mechanism end. The lever can be arranged such that the line connecting the radiator end and the pivoting point is perpendicular to the line connecting the pivoting point and the mechanism end. The pivoting axis may be oriented such that the pivoting axis is perpendicular to the plane through the pivoting point and the two end portions of the lever. The lever may be suspended from the suspension body such that the line connecting the pivoting point and the mechanism end is perpendicular to the support face in a reference state of the lever. By using such a pivoting lever, a movement of the mechanism end is substantially parallel to the support face. Because the lever is rigid, the movement of the mechanism end is transmitted into a movement of the radiator end transversal to the support face. For this reason a lever is advantageous to transmit a movement in a first direction, e.g. parallel to the support face and transversal to the direction of the pivoting axis, into a movement transversal to the first direction. In an advantageous construction the holder comprises three such levers. Each lever having one radiator end, the holder comprises three radiator ends, referred to as a first, a second and a third radiator end. The three levers can be arranged such that the radiator ends define a plane referred to as the radiator plane. The radiator emits its energy along a focusing axis. The focusing axis defines a plane perpendicular to the focusing axis and running through the focal volume, referred to as the focal plane. The radiator can be mounted to the holder such that the focal plane remains parallel to the radiator plane. The focusing axis is now perpendicular to the radiator plane. The first and second radiator ends define a first line in the radiator plane. The third radiator end can be positioned such that the third radiator end is not on the first line. In the remainder of this paragraph it is assumed that translations and rotations are small. If the third radiator end is translated parallel to the focusing axis while at the same time not translating the first and second radiator ends, the radiator plane and the radiator are rotated around the first line, the first line being perpendicular to the focusing axis. This first line is thus a first axis of rotation of the radiator. If the first and second radiator ends are translated over an equal distance parallel to the focusing axis but oppositely directed while at the same time the third radiator end is not translated, the radiator plane and the radiator are rotated around a second axis of rotation. The second axis of rotation runs through the third radiator end and through a fourth point on the first line, said fourth point being the mid point between the first and second radiator end. The second axis of rotation is distinct from the first axis of rotation and perpendicular to the focusing axis. If the first and second radiator ends are translated parallel to the focusing direction but not over an equal distance the fourth point is positioned somewhere on the first line but not necessarily between the first and the second radiator end. If the first, the second and the third radiator end are translated over the same distance and in the same direction, the orientation of the radiator plane remains unaltered and the radiator is moved along the focusing axis. It can be concluded that three levers are advantageous because two independent rotations and a translation of the energy radiator can be realized while minimizing the mechanical strain of the energy radiator.

An embodiment of the apparatus according to the invention is defined in that the second transmission subunit comprises three mechanisms, each mechanism cooperating with one of the three levers and each lever moveably connected to one of the three mechanisms. The advantage of coupling a separate mechanism to each lever is that no clutches are needed to have more than one lever being operated by just one transmission.

An embodiment of the apparatus according to the invention is defined in that the transmission driver unit comprises three further transmission drivers, each mechanism being coupled to one of the three further transmission drivers and each of the three further transmission drivers being coupled to one of the three mechanisms. The advantage of coupling a separate transmission driver to each mechanism is that no clutches are needed to have more than one mechanism being driven by only one driver.

The invention also relates to a MRI device provided with the apparatus according to the invention for thermal treatment of tissue.

### BRIEF DESCRIPTION OF THE DRAWINGS

These and other aspects of the apparatus according to the invention will be exemplarily elucidated and described with reference to the drawings, in which:

FIG. 1 is a perspective view, schematically depicting an embodiment of the MRI device according to the invention.

FIG. 2 is a schematic representation of an embodiment of the apparatus according to the invention.

FIGS. 3 to 14 are schematic representations of portions of the embodiment depicted in FIG. 2.

FIG. 15 is a cross-section according to XV-XV in FIG. 5.

FIG. 16 is a schematic representation of a portion of the embodiment depicted in FIG. 2.

### DETAILED DESCRIPTION OF EMBODIMENTS

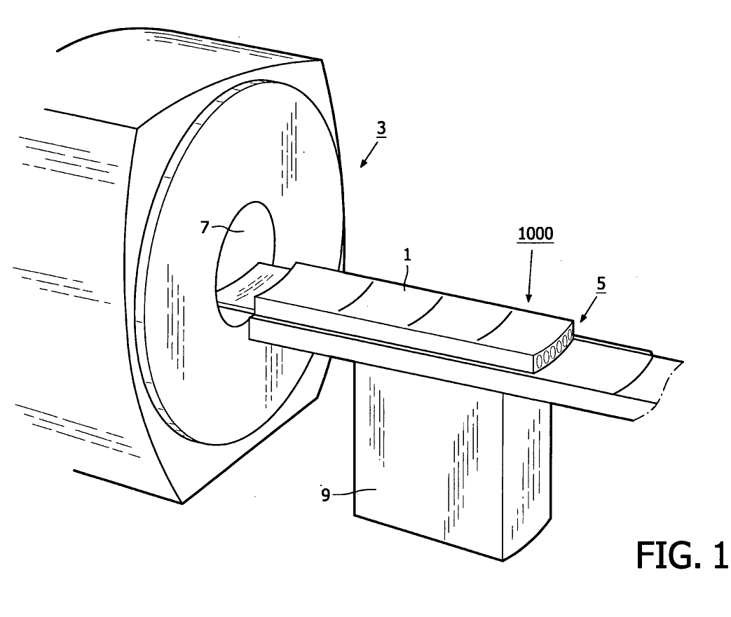

In FIG. 1 an embodiment of a device for Magnetic Resonance Imaging (MRI) 3 according the invention is schematically depicted. The bore 7 of the MRI device 3 is visible and indicated. The MRI device 3 comprises a table 9 and an embodiment of the apparatus according to the invention 1000. The apparatus 1000 comprises a support face 1 for supporting at least a portion of a patient's body and a transmission driver unit 5. One or more step motors, wheels for manual operation, drivable racks cooperating with rods, hydraulic pumps and the like can be mentioned as possible embodiments of the transmission drivers comprised by the transmission driver unit 5. The apparatus 1000 can be shifted from a table 9 into the narrow space of bore 7 of the device for magnetic resonance imaging (MRI) 3. The space available for a patient positioned on the support face 1 is limited to the space within the bore 7 above the support face 1. The space available for an energy radiator and a device for positioning the energy radiator is limited to the space within the bore 7 underneath the support face 1. To relieve the patient possibly suffering from claustrophobia as much as possible the space occupied by the positioning device of the invention should be kept as small as possible. The transmission driver unit 5 should be accessible outside the bore and electrical devices possibly comprised by the transmission driver unit 5 should remain outside the bore 7 to prevent possible interference of fields generated by the transmission drivers of transmission driver unit 5 with the magnetic field used by the device for magnetic resonance imaging 3.

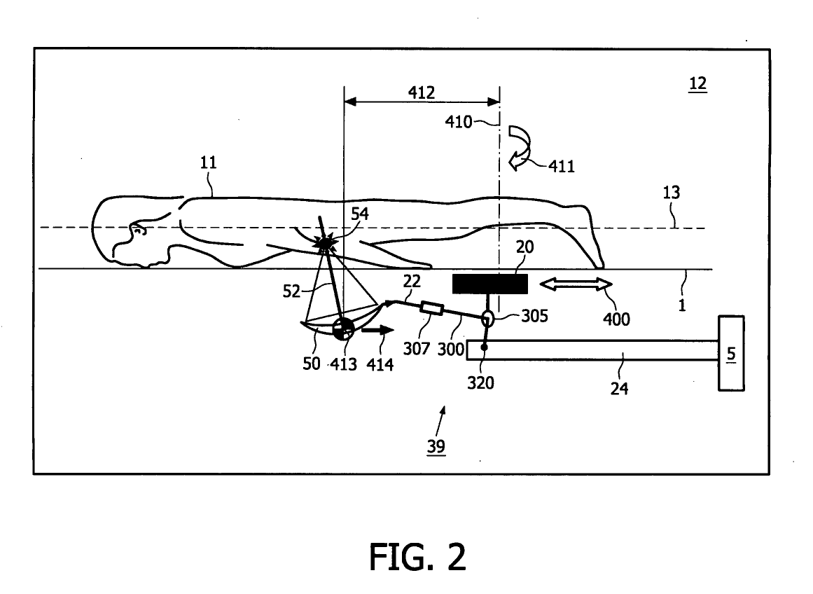

FIG. 2 shows the apparatus according to the invention in a plane of cross-section perpendicular to the support face 1. The plane of cross-section is parallel to the median plane 12 and perpendicular to the support face 1. The support face 1 and the coronal plane 13 of the patient's body 11 are assumed to be parallel, but different positions of the patient's body 11 relative to the support face are possible. The position of the patient's body 11 as exemplarily depicted in FIG. 2 is not limiting the scope of application of the invention. An energy radiator 50 is mounted on a holder 22. The holder 22 is suspended from a suspension body 20. The holder 22 comprises a lever 300. The lever 300 comprises a mechanism end 320. In its mechanism end 320 the lever 300 is rotatably connected to a mechanism 24. The energy radiator 50 directs energy along a focusing axis 52 into a focal volume 54. The direction of the focusing axis 52 can be decomposed in a direction parallel to the coronal plane 13 and a direction perpendicular to the coronal plane 13. The energy is radiated in a direction that is transverse to the coronal plane 13. The orientation of the energy radiator 50 determines the orientation of the focusing axis 52 and the position of the energy radiator 50 determines the position of the focal volume 54. The suspension body 20 can be shifted according to a translation of the suspension body 400 and rotated according to a rotation of the suspension body 411 around an axis of rotation of the suspension body 410, the axis of rotation 410 being transverse to the support plane 1 and the coronal plane 13. The translation 400 and rotation 411 are in a plane substantially parallel to the support face 1 and the coronal plane 13 and oriented perpendicular to the plane of the drawing. The energy radiator 50, the holder 22, the lever 300 and the mechanism 24 are coupled to the suspension body 20 and are translated according to the translation 400. The energy radiator 50, the holder 22 and the lever 300 also follow the rotation 411 of the suspension body 22. The mechanism 24 enables an additional adjustment of the energy radiator 50 and the holder 22. The additional adjustment is superposed on the translation 400 and rotation 411 of the energy radiator 50 according to the translation 400 and rotation 411 of the suspension body 20. The mechanism 24 is connected to transmission driver unit 5. During treatment of the patient the transmission driver unit 5 remains fixed relative to the support face. For this reason, the end of mechanism 24 connected to a part of the transmission driver unit 5 remains fixed relative to the support face 1 also. The other end of the mechanism 24 translates according to the translation 400 if the lever 300 does not rotate around a pivoting point 305, where the lever 300 is rotatably connected to the suspension body 20. For this reason the mechanism 24 is further arranged to change its length between the lever 300 and the transmission driver unit 5. The energy radiator 50 is positioned at a rotation radius 412 from the axis of rotation 410. As a result of the rotation RS 411 around the axis of rotation 410, the energy radiator 50 describes a circle segment around the axis of rotation 410. The resulting position of the radiator 50 relative to its position before rotation 411 around the axis of rotation 410 can be described by a translation 413 and a translation 414. The translation 413 is perpendicular to the plane of drawing and perpendicular to the median plane 12 of the patient's body 11. The translation 414 is in the plane of the drawing parallel to the direction of translation 400. The holder 22, the lever 300 and the mechanism 24 are included in a second transmission subunit 39. The second transmission subunit 39 is arranged for moving the energy generator 50 along the focusing axis 52 and for rotating the energy radiator 50 around two distinct axes perpendicular to the focusing axis 52. The holder 22 of the embodiment of FIG. 2 comprises one lever 300. If the mechanism 24 lengthens while the suspension body 20 and the transmission driver unit 50 remains fixed relative to the support face 1, the mechanism end 320 of lever 300 moves away from unit 5 in a direction substantially parallel to 400. Lever 300 is rotatably suspended from the suspension body 20 in the pivoting point 305. As a result lever 300 rotates around a center of rotation 305 while its mechanism end 320 moves away from driver unit 5. Due to the rotation of lever 300 around center of rotation 305, the holder 22 moves in the direction of the support face 1, i.e. transversal to the coronal plane 13. If the focal volume 54 should describe a translation perpendicular to the support face 1 while orientation of the focusing axis 52 remains unaltered, the displacement of holder 22 has no component parallel to support face 1. For this reason the lever 300 comprises a compliant part 307 to allow for the change in distance between center of rotation 305 and the holder 22. Further means for positioning and orienting the energy radiator 50 are not indicated in FIG. 2 but will be elucidated later. Said further means may be incorporated into the lever 300, but they can also be provided by a different arrangement of the holder 22.

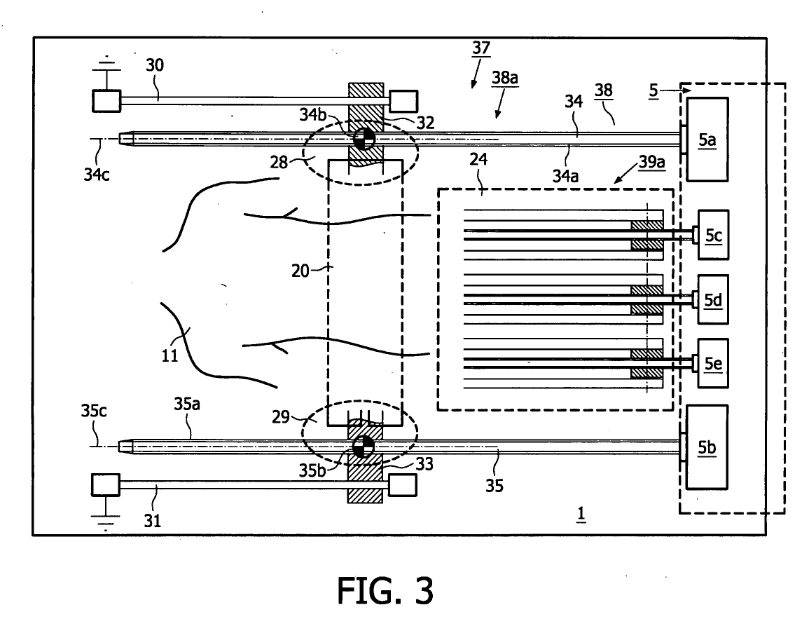

FIG. 3 shows the apparatus according to the invention in a plane parallel to the coronal plane of a patient's body 11. A manipulator 37 comprises a manipulator transmission unit 38a including the suspension body 20, the transmission driver unit 5, mounted to the manipulator transmission unit 38a and the holder 22 not visible in FIG. 3. The plane of the cross-section of FIG. 3 is parallel to the support face 1. The transmission driver unit 5 comprises five transmission drivers 5a, 5b, 5c, 5d and 5e. Transmission driver 5a and 5b are mounted to a first transmission subunit 38. Transmission driver 5c, 5d and 5e are mounted to a second transmission subunit 39. The manipulator transmission unit 38a comprises both the first transmission subunit 38 and the second transmission subunit 39. The mechanism 24 is partly indicated and an embodiment of mechanism 24 will be explained later. The suspension body 20 is partly visible. The energy radiator 50 suspended from the suspension body 20 is not visible in FIG. 3. The suspension body 20 comprises distant portions 28 and 29. The distant portions 28 and 29 are rotatably connected to moveably guided abutments 32 and 33. Distant portion 28 rotates around an axis of rotation 34b relative to moveably guided abutment 32. Distant portion 29 rotates around an axis of rotation 35b relative to moveably guided abutment 33. Transmission bodies 34 and 35 are connected to transmission driver 5a and 5b respectively and cooperate with the moveably guided abutments 32 and 33 respectively. In the embodiment of FIG. 3 the transmission bodies 34 and 35 comprise threaded portions 34a and 35a respectively. The threaded portions 34a and 35a each have a length axis respectively 34c and 35c. The first transmission subunit 38 comprises the moveably guided abutments 32 and 33, the abutment guides 30 and 31 and the transmission bodies 34 and 35. Abutment guides 30 and 31 guide the moveably guided abutments 32 and 33 and are loaded by the weight of the suspension body and components suspended from the suspension body 20. The abutment guides 30 and 31 are fixed relative to the support face 1. The abutment guides 30 and 31 of the embodiment of FIG. 3 are elongated bodies. These elongated bodies should preferably be stiff to minimize their deformation. Stiff abutment guides 30 and 31 can be construed by adapting the shape and the dimensions of the cross-section of the abutment guides 30 and 31 to obtain geometrically stiff bodies. This however is not always possible, because it leads to rather voluminous components. Use can be made of materials having magnetic properties that are compatible for use with magnetic equipment and have a high Young's modulus or modulus of elasticity. Among the ceramic materials the material category of oxides comprises materials as Aluminium oxide (Al2O3) and zinc oxide (ZnO) suitable for use in the abutment guides 30 and 31. Also materials from the category of carbides can be used. Silicon carbide (SiC) is an example of a suitable material that is commercially available. The position of the axes of rotation 34b and 35b of suspension body 20 relative to abutments 32 and 33 respectively is important for forces and torques exerted between the abutment 32 and 33 and the transmission bodies 34 and 35 respectively. For geometrical stiffness it is advantageous if rotation axes 34b and 35b intersect with length axes 34c and 35c respectively.

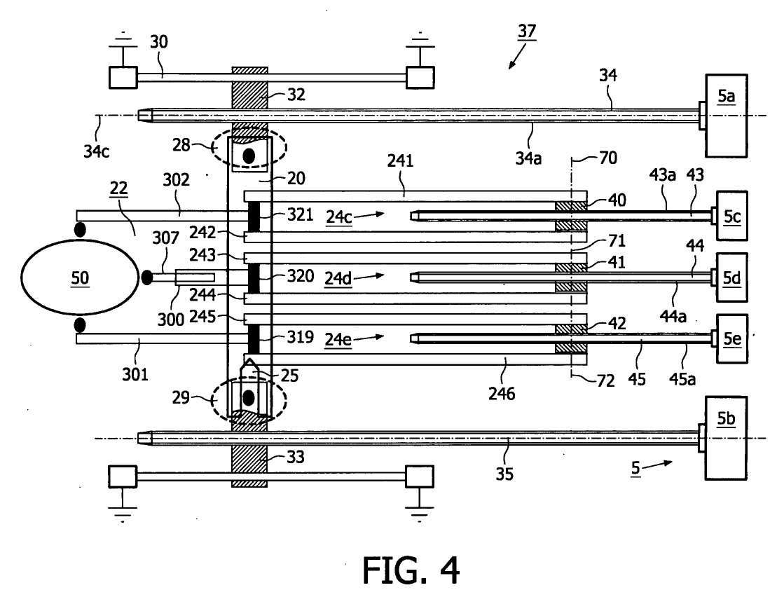

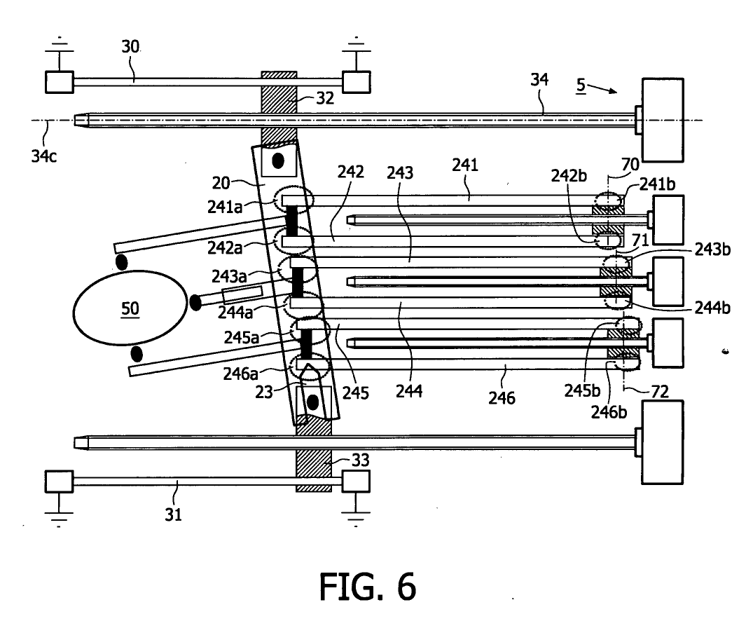

An embodiment of the manipulator 37 according to the invention is schematically depicted in FIG. 4 in a plane parallel to the support face 1. The holder 22 comprises three levers 300, 301 and 302. The levers 300, 301 and 302 are rotatably suspended from the suspension body 20 and are rotatably connected to three separate mechanisms 24d, 24e and 24c in mechanism ends 320, 319 and 321 respectively. The mechanism 24c, 24d and 24e comprise connecting rods 241 to 246, moveably guided bodies 40, 41 and 42 and transmission elements 43, 44 and 45 and mechanism guides 46, 47 and 48, not indicated in FIG. 4. The transmission elements 43, 44 and 45 comprise threaded portions 43a, 44a and 45a. The levers 241 to 246 are rotatably connected to the moveably guided bodies in transmission ends. Axes of rotation 70, 71 and 72 characterize the connection between said levers and moveably guided bodies. The moveably guided bodies 40, 41 and 42 cooperate with the threaded portions 43a, 44a and 45a of rotatable transmission elements 43, 44 and 45. The transmission elements 43, 44 and 45 are each connected to a transmission driver 5c, 5d and 5e respectively. The interaction between the mechanisms 24c, 24d and 24e and the lever 302, 300 and 301 respectively is according to the interaction of lever 300 of FIG. 2 with mechanism 24 of FIG. 2 and as explained above. The holder 22 and the energy radiator 50 are positioned and oriented relative to the suspension body 20 as a result of the displacement of the mechanism ends 321, 320 and 319. To maintain the position and orientation of the holder 22 and the energy radiator 50 relative to the suspension body 20, the mechanism ends 321, 320 and 319 of said levers should maintain their position relative to the suspension body 20. The mechanism ends 321, 320 and 319 being fixed relative to the connecting rods 241 to 246 or at least to first end portions 241 a to 246a (indicated in FIG. 6) of the connecting rods 241 to 246, said connecting rods should maintain their position relative to the suspension body 20. If the suspension body 20 is translated and rotated in the plane of the drawing as a result of a rotation of transmission driver 5a and 5b, the mechanism ends 321, 320 and 319 and the first end portions 241a to 246a (FIG. 6) are translated according to the translation and rotation of the suspension body 20. To keep the position and orientation of the holder 22 and the energy radiator 50 unaltered with respect to the suspension body 20, the moveably guided bodies 40, 41 and 42 and second end portions 241b to 246b (FIG. 6) of the connecting rods 241 to 246 positioned around the axes of rotation 70, 71 and 72 should be translated according to the translation and rotation of the suspension body 20. For this reason, the transmission drivers 5c, 5d and 5e should compensate for a translation and a rotation of the suspension body 20 in the plane of the drawing by driving the transmission elements 43, 44 and 45 if the position and orientation of the holder 22 and the energy radiator 50 relative to the suspension body 20 are to remain unaltered. The rotations of the transmission drivers 5a and 5b, driving the first transmission subunit 38 are thus coupled to adjustment of the second transmission subunit 39 by transmission driver 5c, 5d and 5d if the position and orientation of the radiator 50 relative to the suspension body 20 is not changed during the translation and the rotation of the suspension body 20.

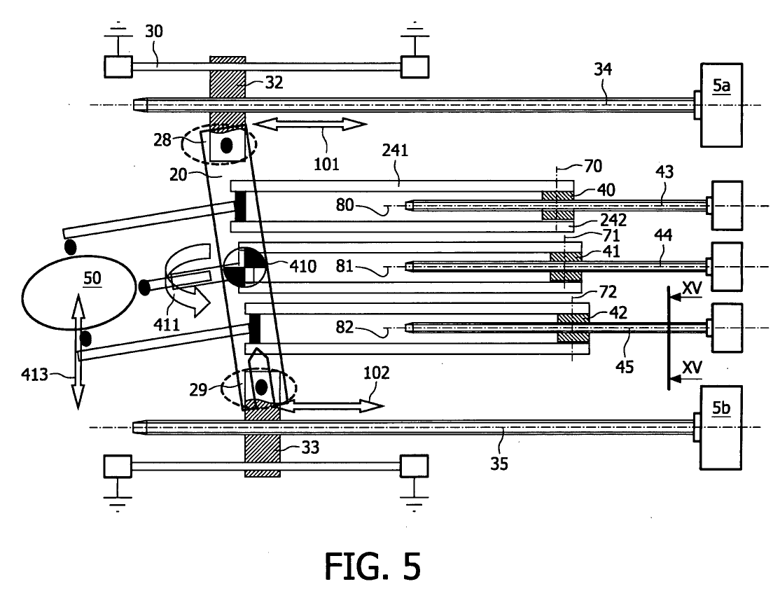

In FIG. 5 the adjustment of the suspension body 20 of an embodiment of the apparatus according to the invention is schematically depicted. Transmission driver 5a drives the rotatable threaded transmission body 34. The transmission body 34 cooperates with moveably guided abutment 32 and as a result of the rotation of the transmission driver 5a the moveably guided abutment 32 and the distant portion 28 of suspension body 20 are translated along abutment guide 30 over a distance 101. Similarly, transmission driver 5b causes a translation of distant portion 29 of the suspension body 20 over a distance 102. In general, the distance 101 will not be equal to distance 102 and the suspension body will rotate around the axis of rotation 410. As a result of this rotation 411 around axis of rotation 410 the energy radiator 50 will be positioned by a translation 413 transversal to abutment guide 32 and in the plane of the drawing. Also shown in FIG. 5 are the transmission elements 43, 44 and 45. The transmission elements of the exemplary embodiment as depicted in FIG. 5 comprise threaded portions 43a, 44a and 45a (FIG. 4). The threaded portions each have a length axis 80, 81 and 82. The connecting rods 241 to 246 are rotatably connected to the moveably guided bodies 40, 41 and 42, having axes of rotation 70, 71 and 72. It is advantageous for geometrical stiffness if the length axes 80, 81 and 82 intersect with the axes of rotation 70, 71 and 72. In that situation the mechanisms 24c, 24d and 24e are designed as push and pull mechanisms. Shear forces and bending moments, causing significant deformation of the transmission elements 43, 44 and 45 and causing a bad contact situation by tilting the moveably guided bodies can be minimized. A further advantage of the embodiment of FIG. 5 is that the transmission element 40 is symmetrically loaded by connecting rods 241 and 242. Due to the symmetric arrangement of the connecting rods around the transmission element 40, bending moments in the transmission element 40 and deformation of the transmission element 40 will be minimized. The symmetrical arrangement of rods 241 and 242 around axis 80 prevents tilting of the moveably guided body 40 around a tilting axis perpendicular to the plane through axes 70 and 80. The same applies to the topology of transmission elements 41 and 42 relative to the connecting rods attached to the transmission elements 41 and 42. Again hysteresis is minimized for good positioning accuracy.

In an embodiment according to the invention as schematically depicted in FIG. 6 the mutual influencing between the positioning of connecting rods 241 to 246 on the one side and the position of the moveably guided abutments 32 and 33 on the other side is illustrated for another position and rotation of the suspension body 20. Again it is assumed that the position and orientation of the energy generator 50 relative to the suspension body 20 is the same as in FIG. 4 and FIG. 5, implying that the position of the connecting rods 241 to 246 (and in particular first end portions 241 a to 246 a of said rods 241 to 246) relative to the suspension body 20 is the same as in FIG. 4 and FIG. 5. The suspension body 20 is positioned close to the transmission driver unit 5. The second end portions 241b to 246b of connecting rods 241 and 242 are almost at their ultimate position to compensate for the position of the suspension body 20 along the abutment guides 30 and 31. The suspension body 20 is rotated relative to the length axis 34c of transmission body 34. To compensate for this rotation the rotation axes 70, 71 and 72 of connecting rods 241 to 246 are not aligned. The suspension body 20 is rotatably connected to the moveably guided abutments 32 and 33. The distance between the moveably guided abutments 32 and 33 changes according to the rotation of the suspension body 20. A slot 23 is provided to the suspension body 20 to allow for this varying distance and to enable a stress-free rotation of the suspension body 20.

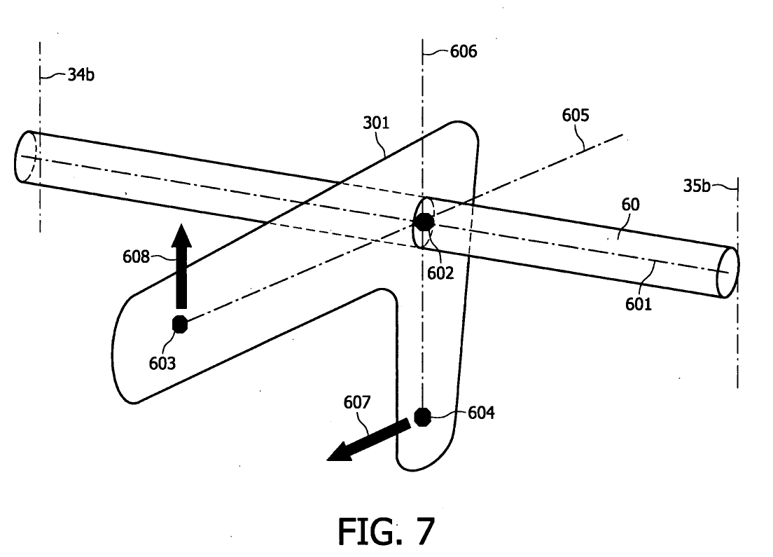

An embodiment of the lever 301 or 302 comprised by the holder 22 as indicated in FIG. 4 is schematically shown in FIG. 7 in elevated view. The lever 301 (or 302) is rotatably suspended from an axle 60 with length axis 601. The axle 60 is fixed relative to the axes of rotation 34b and 35b (see also FIG. 3), being the axes of rotation of the suspension body 20 with respect to the moveably guided abutments 32 and 33 (not indicated in FIG. 7). The lever 301 is rotatably suspended from the suspension body such that it can pivot around a pivoting point 602 on a pivoting axis 601 being the length axis of axle 60. The lever 301 has two more characterizing points or lever ends 603 and 604, the lever ends being different from the pivoting point 602. In the lever ends the lever 301 is connected to other components. One end 603 of the lever 301 is rotatably connected to the radiator 50 (not indicated in FIG. 7), while the other end 604 is rotatably connected to the connecting rods 245 and 246 of mechanism 24e (not indicated in FIG. 7). The end of the lever, connected to the radiator 50 will be referred to as the radiator end 603. The end of the lever 301, connected to the mechanism 24e comprised by the second transmission subunit 39 will be referred to as the mechanism end 604. Line 605 connects the radiator end 603 and the pivoting point 602. Line 606 connects the mechanism end 604 and the pivoting point 602. The lever 301 is arranged such that line 605 is perpendicular to line 606. The pivoting axis 601 is oriented such that it is perpendicular to lines 605 and 606. In the remainder of this paragraph it is assumed that line 606 is substantially perpendicular or transversal to the support face 1 (not indicated in FIG. 7). Than, the support face 1 is substantially parallel to the plane defined by pivoting axis 601 and line 605. In the remainder of this paragraph it is assumed that translations and rotations are small. The connecting rods 245 and 246 can impose a translation 607 on mechanism end 604. Translation 607 is parallel to line 605 and substantially parallel to the support face 1. Because the lever 301 is rigid, translation 607 is transmitted into a translation 608 of the radiator end 603. Translation 608 is parallel to line 606 and substantially perpendicular or transversal to the support face 1. It can thus be concluded that lever 301 transmits a translation 607 of mechanism end 604 into a translation 608 of radiator end 603, translation 607 having a component, which component is perpendicular to translation 608.

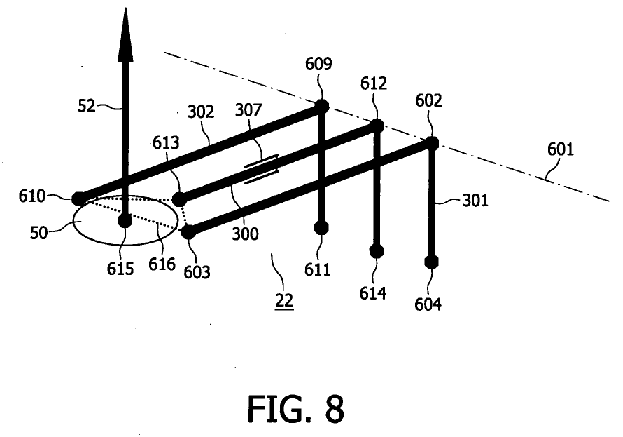

In an embodiment of the holder 22 according to the invention as schematically depicted in FIG. 8 the holder 22 comprises three levers 300, 301 and 302. The levers 300, 301 and 302 comprise radiator ends 613, 603 and 610 respectively and mechanism ends 614, 604 and 611 respectively. The levers 300, 301 and 302 have the axis of rotation 601 parallel to the support face 1 as a common axis of rotation. The levers 300, 301 and 302 can pivot around pivoting points 612, 602 and 609 respectively. The arrangement and the operation of the lever 300, 301 or 302 is similar as explained according to FIG. 7. Lever 300 however has a length change device or compliant portion 307 as explained in relation to FIG. 2. Center of rotation 305, indicated in FIG. 2, corresponds with pivoting point 612 and pivoting axis 601 as indicated in FIG. 6. Compliant portion 307 is compliant along the line connecting radiator end 613 and pivoting point 612. In FIG. 6 also the energy radiator 50 and the focusing axis 52 are indicated. The energy radiator 50 can e.g. be mounted to the levers 300, 301 and 302 by ball-joints. The focusing axis intersects a face of the energy radiator 50 in a radiator face point 615. The radiator ends 603, 610 and 613 together form a triangle 616. The energy radiator is mounted to the levers 300, 301 and 302 in the radiator ends 603, 610 and 613. The energy radiator 50 is a rigid body. For this reason the triangle 616 remains its shape irrespective of the orientation of the levers 300, 301 and 302. The focusing axis 52 is perpendicular to the triangle 616. In FIG. 8 the holder 22 and the energy generator 50 are represented in a reference state with respect to the support face 1. In the reference state the triangle 616 is parallel to the support face 1 and the focusing axis 52 is transversal to the support plane 1. Radiator ends 610, 613 and 603 can be translated in a direction parallel to the direction of the focusing axis 52 by rotation of the levers 302, 300 and 301 respectively.

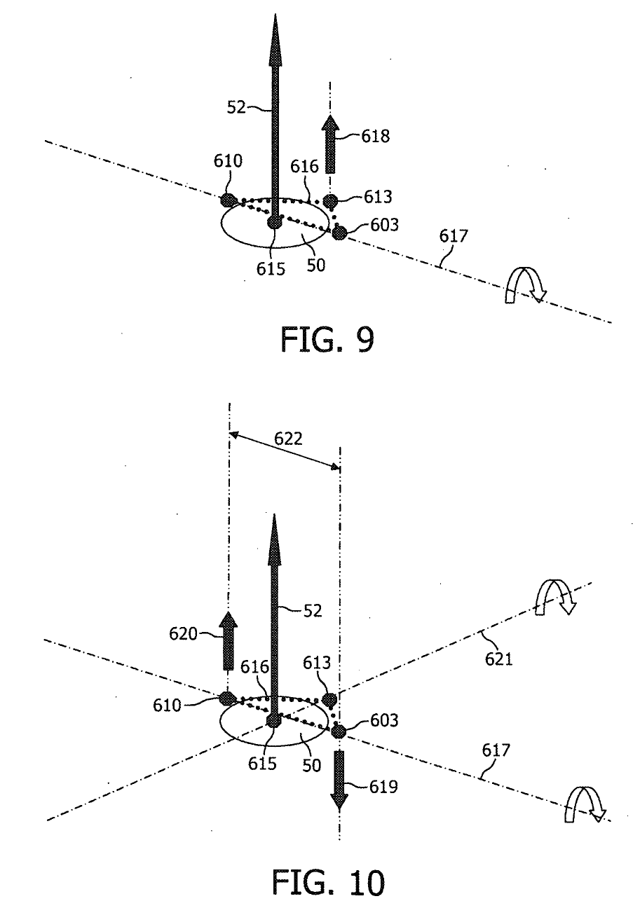

In FIG. 9 a detail of the embodiment as described in FIG. 8 is shown. It is assumed that radiator ends 603 and 610 are not translated and that the levers 301 and 302 to which these ends are connected (FIG. 8) are not rotated, i.e. the levers 301 and 302 stay in their reference state. A rotation of lever 300 around axis of rotation 601 (FIG. 8) will result in a small translation or displacement 618 of radiator end 613. Actually, radiator end 613 describes a small circle segment around an axis of rotation 617. The direction of this displacement 618 is parallel to the focusing axis 52 of the energy generator 50. As a result the triangle 616 and the radiator 50 will rotate around a first axis of radiator rotation 617. The radiator ends 603 and 610 remain in the reference position relative to the pivoting axis 601 (FIG. 8). A rotation of the energy radiator 50 around axis of rotation 617 will result in enlargement of the distance between pivoting point 612 (fixed relative to the suspension body 20) and radiator end 613 (FIG. 8). For this reason the lever 300 comprises the compliant portion 307 (FIG. 8).

In FIG. 10 a detail of the embodiment as described in FIG. 8 is shown. It is assumed that radiator end 613 is not translated and that the lever 300 to which this end is connected (FIG. 8) is not rotated, but that lever 300 is still in its reference state. Opposite rotations of equal magnitude of identical levers 301 and 302 around axis of rotation 601 (FIG. 8) will result to opposite displacements 619 and 620 of equal magnitude of radiator ends 603 and 610 respectively. The direction of said displacements 619 and 620 is parallel to the direction of the focusing axis 52 of the energy generator 50. As a result the triangle 616 and the radiator 50 will rotate around a second axis of radiator rotation 621. The levers 300, 301 and 302 can pivot around pivoting points 612, 602 and 609 respectively (FIG. 8). The pivoting points 612, 602 and 609 are on the pivoting axis 601 (FIG. 8). The second axis of rotation 621 runs through pivoting point 612 because lever 300 is not rotated from its reference state and the plane of triangle 616 comprises the second axis 621. Radiator points 610 and 603 keep their distance 622 because they are attached to the rigid energy radiator 50. After rotation of the energy radiator 50 around the second axis of rotation 621 the projection of distance 622 on pivoting axis 601 along a direction perpendicular to the pivoting axis 601 is shorter than the distance between the pivoting points 602 and 609 (FIG. 8) in the reference state. This effect causes a deformation of the levers 301 and 302 (FIG. 8). Radiator ends 610 and 603 of levers 301 and 302 bend towards each other as a result of a rotation around the second axis of rotation 621. This deformation may cause damage to the energy generator 50 because bending forces and moments are introduced as a result of the deformation of levers 301 and 302. To absorb this deformation, the levers should be compliant in the direction of the deformation, being the direction perpendicular to the second axis of rotation and parallel to the support face 1 in the reference state of the holder 22.

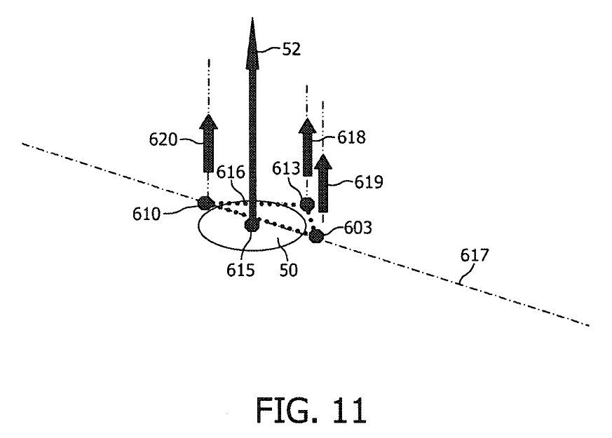

In FIG. 11 a detail of the embodiment as described in FIG. 8 is shown. If the levers 300, 301 and 302 are rotated such that the resulting translation 618, 619 and 620 of radiator ends 613, 603 and 610 are equal and in the same direction, the energy radiator 50 is translated along its focusing axis 52. Relative to the suspension body 20 and pivoting axis 601 (FIG. 8), the energy radiator 50 describes a circle segment while remaining its orientation relative to the suspension body 20.

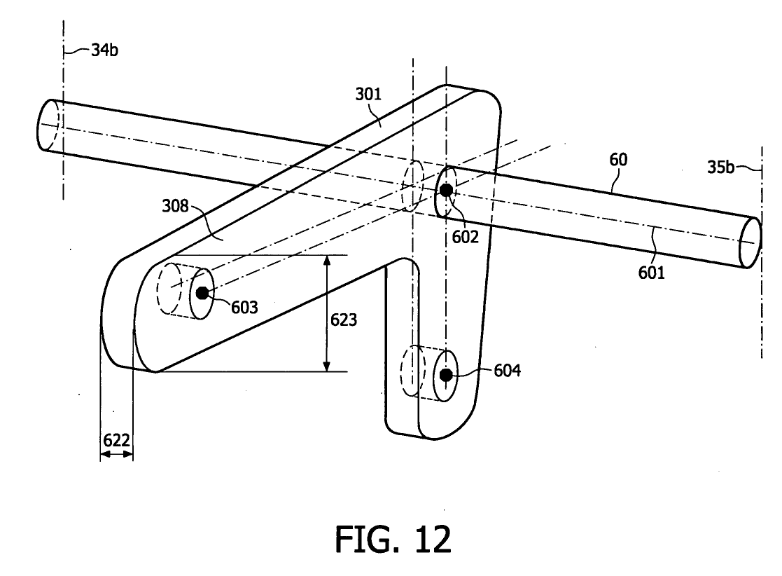

In FIG. 12 an embodiment of lever 301 is schematically depicted. The lever 301 is compliant in the direction of pivoting axis 601 and stiff in the direction of axes 34b and 35b through the distant portions 28 and 29 of the suspension body 20 (FIG. 3). The lever 301 is also stiff in a direction perpendicular to axes 601 and 34b. The stiffness distribution is determined e.g. by height 623 and thickness 622 of the lever 301. The lever 301 as schematically depicted in FIG. 12 has a portion 308. The portion 308 is compliant in the direction parallel to axis 601. To achieve compliance of lever 301 along the direction of pivoting axis 601, the pivoting point 602 could be implemented as a slideable hinge along axis 601.

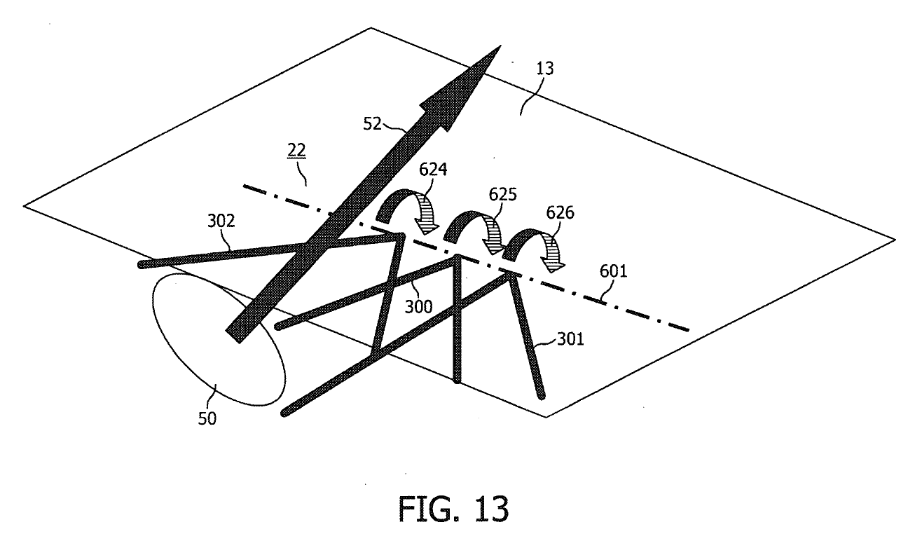

In an embodiment of the holder 22 according to the invention as schematically depicted in FIG. 13 the holder 22 comprises three levers 300, 301 and 302. Due to rotations 624, 625 and 626 of levers 300, 301 and 302 the energy radiator 50 and the focusing axis 52 are positioned and oriented relative to the coronal plane 13.

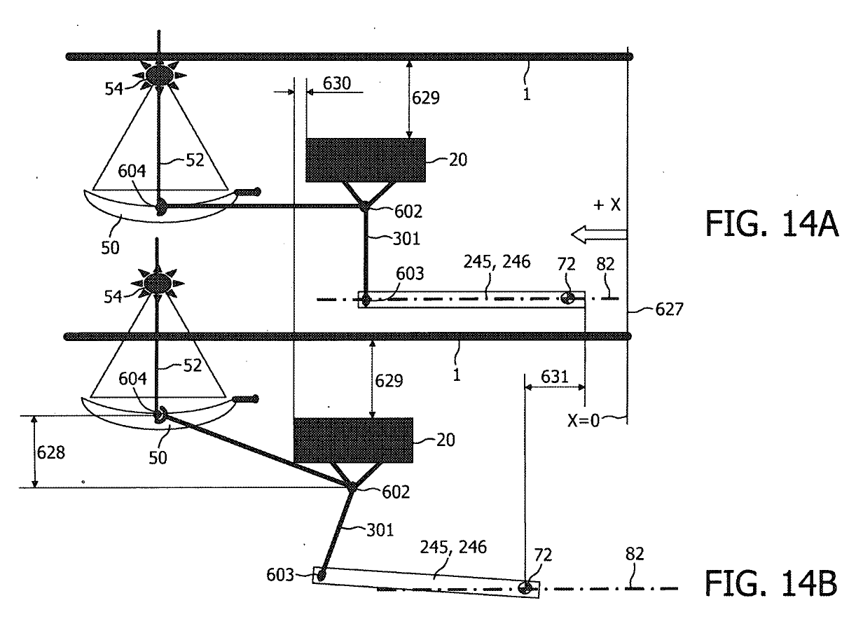

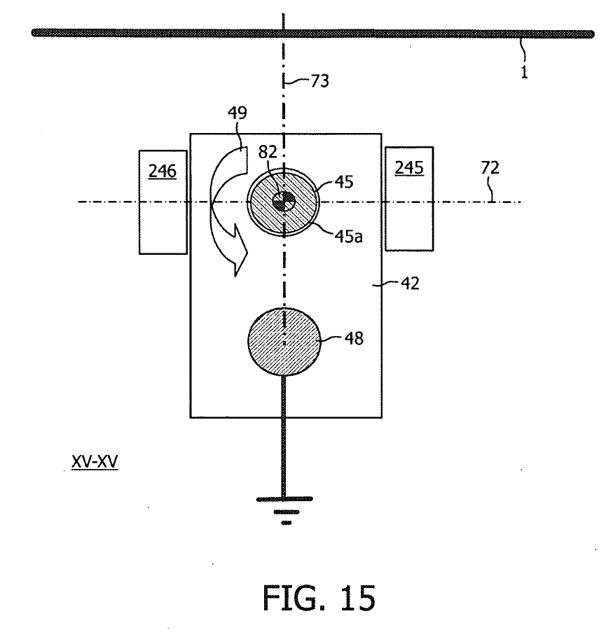

In FIG. 14a a detail of the embodiment according to the invention as described in FIG. 8 is shown in its reference state relative to the support face 1 and relative to a line of reference X=0 627 perpendicular to the support face 1. In FIG. 14b the same detail as in FIG. 14a is depicted but in a state different from the reference state. The energy radiator 50 and the focusing axis 52 as shown in FIG. 14a have the same orientation relative to the support face 1 and the suspension body 20 as shown in FIG. 14b. The only difference between the energy radiator 50 of FIG. 14a and FIG. 14b is, that the position of the energy radiator 50 relative to the suspension body 20 and the support face 1 is shifted over a vertical translation 628 of the energy radiator 50. In the direction parallel to the support face 1 and in the plane of the drawing, indicated as the X-direction, the energy radiator 50 is not translated and the distance of the focal volume 54 to the line of reference 627 as depicted in FIG. 14 a is the same in FIG. 14b. To realize the vertical translation 628 of the energy radiator 50, the lever 301 rotates around pivoting 602. Pivoting point 602 is fixed relative to the suspension body 20 and the vertical distance between the suspension body and the support face 629 is constant because the abutment guides remain fixed and parallel to the support face (see description of FIG. 3). Lever 301 is rigid, so the distance between pivoting point 602 and radiator end 604 remains unaltered during rotation of the lever 301. For this reason the suspension body 20 must be repositioned parallel to the X direction over a correction distance 630. The connecting rods 245 and 246 are shifted and reoriented as a result. The connecting rods are rotatably connected to the mechanism end 603 of the lever 301 and are rotatably connected to the moveably guided body 45 in axis of rotation 72. Axis of rotation 72 intersects with the length axis 82 of the threaded portion 45a (FIG. 4) of the transmission element 45 (FIG. 4). The position and orientation of the length axis 82 is fixed with respect to the support face 1. As a consequence the connecting rods 245 and 246 describe a combined translation and rotation during rotation of the lever 301. The motion of the connecting rods 245 and 246 arises from the restrictions that one end of the connecting rods 245 and 246 is rotatably connected to the mechanism end 603 of lever 301 and rotates around pivoting point 604 while the other end is rotatably connected to the moveably guided body 42 in a point on axis 72 and translates along the direction of axis 82. The translation 631 of the axis of rod rotation 72 relative to the moveably guided body 42 is indicated in FIG. 14. The transmission driver 5e realizes translation 631, while the transmission drivers 5a and 5b realize the translation of the suspension body 630. It is thus illustrated that a translation of the energy radiator 50 towards the support face 1 and a matching repositioning of the focal volume 54 can be realized by a coupled and coordinated action of several transmission drivers 5a, 5b and 5c. Mechanism guide 48 as schematically depicted in FIG. 15 prevents the rotation of the moveably guided body 42 around the length axis 82.

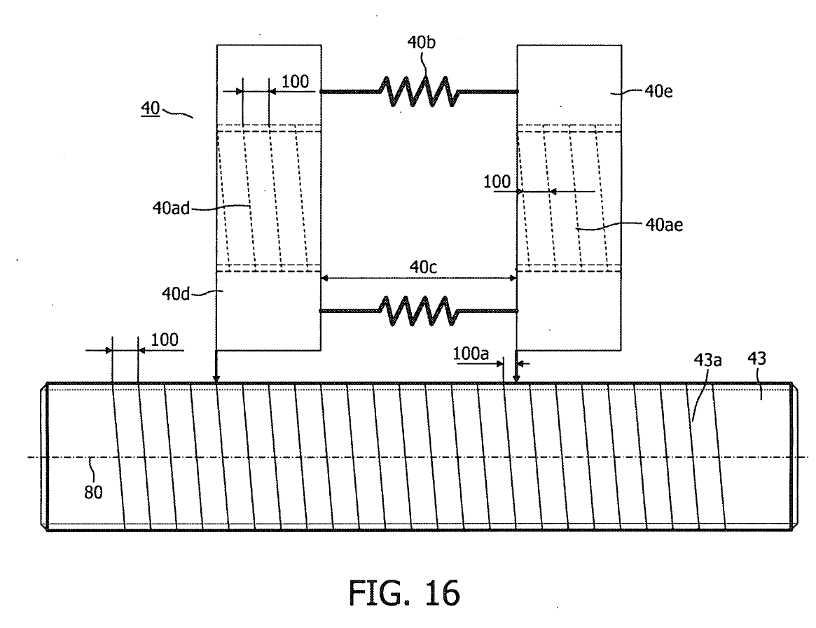

In FIG. 15 an embodiment of part of the second transmission is schematically shown. The cross-section depicted in FIG. 15 is according to view XV-XV as indicated in FIG. 5. The connecting rods 245 and 246 are rotatably connected to moveably guided body 42. Axis of rotation 72 intersects with the length axis 82 of a threaded portion 45a of rotatable transmission element 45. Friction between the threaded portion 45a of transmission element 45 and the cooperating internal portion of the moveably guided body 45 exerts a friction moment 49 on moveably guided body 45 around the length axis 82 of its threaded portion 45a. A mechanism guide 48 prevents a rotation of the moveably guided body 42 according to the friction moment 49. Mechanism guide 48 is parallel to the support face 1. In the embodiment of FIG. 15 only one mechanism guide 48 is guiding the body 42. More guides can be applied to obtain even higher stiffness of the second transmission unit. The connecting rods 245 and 246 are symmetrically arranged around the moveably guided body 42 and the length axis 82 to prevent bending of the transmission element 45 and tilting of the moveably guided body 45 around tilting axis 73. The tilting axis 73 is perpendicular to the support face 1 and to the axis of rotation 72. Similarly, mechanism guides 47 and 48 (not indicated) may be provided to prevent a rotation of the moveably guided bodies 40 and 41 respectively. In FIG. 16 an embodiment of moveably guided body 40 of the second transmission subunit is schematically shown. The moveably guided body 40 is depicted disassembled from the rotatable threaded transmission element 43. In assembled state the moveably guided body 40 can be moved by rotation of element 43. The moveably guided body 40 comprises a compliant portion 40b and two rigid portions 40d and 40e. The rigid portions 40d and 40e are provided with an internal thread 40ad and 40ae respectively. Internal threads 40ad and 40ae correspond to the threaded portion 43a of rotatable transmission element 43. The threads 40ad, 40ae and 43a are provided with a corresponding pitch 100. The compliant portion 40b of moveably guided body 40 has a length 40c in a state, wherein the moveably guided body is not mounted to the transmission element 43. The length 40c of compliant portion 40b differs by a distance 100a from zero or more whole pitches 100. For this reason, the compliant portion 40b will be deformed over a distance of at least 100a when it is mounted to the transmission element 43 in a compressed state. The compliant portion 40b can also be expanded over a distance of at least pitch 100 minus distance 100a. More pretension can be introduced by compression or expansion of compliant portion 40b over more than one pitch 100. Pretension can be introduced elsewhere in the apparatus according to the embodiment as depicted in FIG. 16.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single mechanism or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

1. An apparatus for thermal treatment of tissue, provided with a support face for supporting at least a portion of a patient's body and comprising:
an energy radiator mounted to a holder, for directing energy along a focusing axis into a focal volume, and
a manipulator comprising:
a manipulator transmission unit including a suspension body,
a transmission driver unit mounted to the manipulator transmission unit, the transmission driver unit comprising at least one transmission driver and
the holder suspended from the suspension body, wherein the manipulator transmission unit is driveable by the transmission driver unit and the holder is driveable by the manipulator transmission unit, wherein the manipulator transmission unit has a first transmission subunit for translating and rotating the suspension body in a plane substantially parallel to the support face and has a second transmission subunit for moving the energy radiator along the focusing axis and for rotating the energy radiator around two distinct axes perpendicular to the focusing axis.

2. The apparatus as claimed in claim 1, wherein the suspension body comprises distant portions and the first transmission subunit comprises moveably guided abutments and abutment guides, each of the distant portions being rotatably connected to at least one of the moveably guided abutments and each moveably guided abutment being guidably supported by at least one of the abutment guides.

3. The apparatus as claimed in claim 2, wherein the first transmission subunit comprises at least one transmission body for cooperation with one of the moveably guided abutments and being coupled to a separate transmission driver of the transmission driver unit.

4. The apparatus as claimed in claim 3, wherein the distant portions each have a first axis of rotation with respect to the moveably guided abutment to which the respective distant portion is connected and wherein the transmission body cooperating with the respective moveably guided abutment comprises a first threaded portion, said portion having a first length axis, said first length axis intersecting with said first axis of rotation.

5. The apparatus as claimed in claim 4, wherein at least a part of at least one of the moveably guided abutments is elastically deformed to establish pretension between the moveably guided abutment and the first threaded portion comprised by the transmission body cooperating with the respective moveably guided abutment.

6. The apparatus as claimed in claim 1, wherein the holder comprises three levers, moveably suspended from the suspension body.

7. The apparatus as claimed in claim 6, wherein the second transmission subunit comprises three mechanisms, each mechanism cooperating with one of the three levers and each lever being moveably connected to one of the three mechanisms.

8. The apparatus as claimed in claim 7, wherein the transmission driver unit comprises three further transmission drivers, each mechanism being coupled to one of the three further transmission drivers and each of the three further transmission drivers being coupled to one of the three mechanisms.

9. The apparatus as claimed in claim 8, wherein each mechanism comprises a connecting rod, a moveably guided body, a mechanism guide and a transmission element for cooperation with the moveably guided body, the transmission element being driveably connected to the transmission driver coupled to the respective mechanism, each connecting rod comprising a first and a second end portion, each first end portion being rotatably connected to one of the levers and each second end portion being rotatably connected to one of the moveably guided bodies, the respective moveably guided body being guidably supported by the mechanism guide.

10. The apparatus as claimed in claim 6, wherein a portion of at least one of the levers is substantially compliant in a first direction and substantially stiff in a second direction perpendicular to the first direction and wherein at least one of the other levers has a portion substantially stiff in the first direction and substantially compliant in the second direction.

11. The apparatus as claimed in claim 10, wherein the transmission element cooperating with the moveably guided body comprises a second threaded portion for cooperation with the moveably guided body.

12. The apparatus as claimed in claim 11, wherein the transmission element has an axis of rod rotation with respect to the moveably guided body and wherein the second threaded portion of the transmission element has a second length axis the axis of rod rotation intersecting with the second length axis.

13. The apparatus as claimed in claim 12, wherein at least part of each moveably guided body is elastically deformed to establish pretension between the moveably guided body and the second threaded portion comprised by the transmission element cooperating with the moveably guided body.

14. The apparatus as claimed in claim 13, wherein each mechanism comprises at least one connecting rod providing portions symmetrically arranged around the moveably guided body to which the at least one connecting rod is moveably connected.

15. The apparatus according to claim 1, wherein at least one of its components comprises a material, having magnetic properties that are suitable for use in equipment for magnetic resonance imaging.

16. The apparatus according to claim 2, wherein the abutment guides, the mechanism guides and the suspension body comprise ceramic material.
